(19) United States
(12) Patent Application Publication
(10) Pub. No.: US 2015/0056269 A1
(43) Pub. Date: Feb. 26, 2015

(54) LIPOSOME COMPRISING AT LEAST ONE CHOLESTEROL DERIVATIVE

(71) Applicants: Corinne Chaimbault, Marseille (FR); Magali Michaud, Marseille (FR); Patrick Berna, Cassis (FR); Sophie Schaller, Marseille (FR); Firas Bassissi, Dijon (FR); Simon Eastman, Delta (CA)

(72) Inventors: Corinne Chaimbault, Marseille (FR); Magali Michaud, Marseille (FR); Patrick Berna, Cassis (FR); Sophie Schaller, Marseille (FR); Firas Bassissi, Dijon (FR); Simon Eastman, Delta (CA)

(73) Assignee: TROPHOS, Marseille (FR)

(21) Appl. No.: 14/240,990

(22) PCT Filed: Aug. 22, 2012
(86) PCT No.: PCT/FR2012/051924

§ 371 Date: Nov. 10, 2014

(30) Foreign Application Priority Data
Aug. 25, 2011 (FR) 1102594
Sep. 21, 2011 (FR) 1102862

(51) Int. Cl. A61K 31/15; A61K 31/56; A61K 9/127

(52) U.S. Cl. CPC A61K 31/15; A61K 9/127; A61K 31/56
USPC 424/450; 514/640; 514/177

(57) ABSTRACT

The present invention relates to a novel galenic form of cholesterol derivative.

More particularly the invention relates to liposomes comprising at least one cholesterol derivative and compositions comprising said liposomes.

The present invention relates to a novel galenic form of cholesterol derivatives.

More particularly, the invention relates to liposomes comprising at least one cholesterol derivative corresponding to formula (I) described hereafter and the compositions comprising said liposomes.

By way of simplification, the expression “cholesterol derivative” in the present text can be used to denote the compounds of formula (I), or even their homologues which are not specifically described in the present application, as well as their analogues. In fact, all these compounds of formulae I have the cholesterol skeleton in common.

The invention relates to, but is not constituted only by, all these compounds and their homologues not mentioned here, which have structural similarities.

Thus the invention relates to liposomes comprising at least one compound corresponding to the following formula (I)

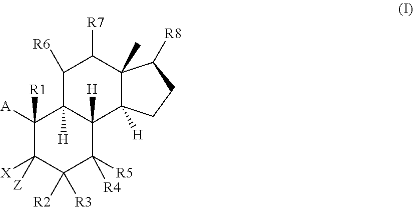

in which,
R₂ can represent a hydrogen atom or a C₁-C₆ alkyl, C₃-C₆ cycloalkyl, C₂-C₆ alkenyl, C₂-C₆ alkynyl, aryl group, a heterocycle, or a halogen atom or a —CN, —CF₃, —NO₂, —OR^{a}, —SR^{a}, —SO₂R^{a}, —NR^{a}R^{b}, —C(O)—R^{a}, —OC(O)R^{a}, —OC(O)NR^{a}R^{b}, —C(O)OR^{a}, —C(O)NR^{a}R^{b} group, in which
   (i) R^{a} and R^{b}, simultaneously or independently of one another, can be chosen from a hydrogen atom or a C₁-C₆ alkyl group, a C₂-C₆ alkenyl group, a C₃-C₆ cycloalkyl group, an aryl group, a heterocyclic group or
   (ii) R^{a} and R^{b} together can form a linear or branched hydrocarbon chain, having 2 to 6 carbon atoms, optionally comprising one or more double bonds and/or optionally interrupted by one or more oxygen, sulphur or nitrogen atom(s), or
   (iii) R^{a} and R^{b} together with the nitrogen to which they are attached can form a C₃-C₆ heterocycle, said heterocycle can comprise one or more double bonds and/or one or more oxygen, sulphur or nitrogen atom(s);
R₃ can represent a hydrogen atom or a C₁-C₆ alkyl group, or
R₂ and R₃ together with the carbon to which they are attached, can represent an oxygen atom or a C₃-C₆ cycloalkyl group or an ═N—OH, ═CH—(C₁-C₆)alkyl, ═CH-aryl, ═CH—(C₃-C₆)cycloalkyl group;
R₄ can represent a hydrogen atom or a C₁-C₆ alkyl group, or a hydroxyamino (—NH₂—OH) group; or
R₃ and R₄ together can form an additional carbon-carbon bond between the carbon atoms to which they are attached, or a C₃-C₆ cycloalkyl group;
R₅ can represent a hydrogen atom or a C₁-C₆ alkyl, C₃-C₆ cycloalkyl, C₂-C₆ alkenyl, C₂-C₆ alkynyl, aryl group, or a —CN, —OR^{a}, —SR^{a}, —SO₂R^{a}, —NR^{a}R^{b}, —C(O)—R^{a}, —OC(O)R^{a}, —OC(O)NR^{a}R^{b} group, R^{a} and R^{b} being as defined previously, or a hydroxyamino (—NH₂—OH) group;
R₈ can represent a group chosen from
   (i) a C₄-C₁₂ alkyl group or a C₄-C₁₂ alkenyl group, in particular a group chosen from

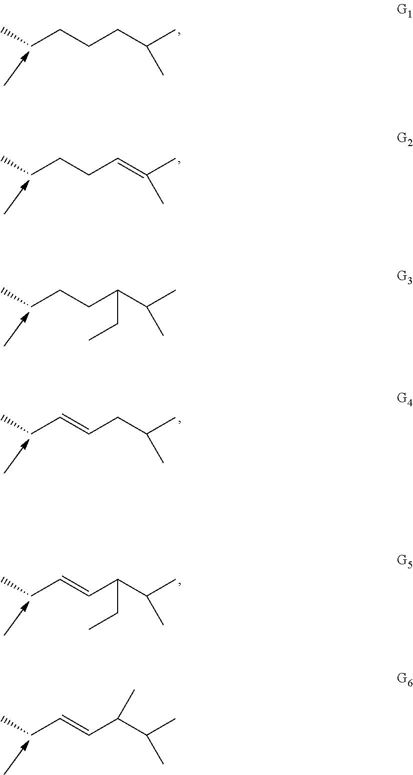

(ii) a group corresponding to formula (II) as follows:

R₁₄—Y—R₁₅ (II)
in which:
   R₁₄ can represent a C₄-C₁₂ alkyl group or a C₄-C₁₂ alkenyl group, in particular a C₆-C₁₀ alkyl, group preferentially the following G₇ group

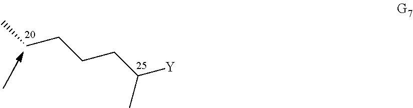

and
Y can represent an oxygen atom or an —NR^{a} group with R^{a} being as described previously, and
R₁₅ can represent a C₁-C₆ alkyl, C₃-C₆ cycloalkyl, aryl, heterocycle, —C(O)—(C₁-C₆)alkyl, —C(O)—(C₃-C₆)cycloalkyl, —C(O)-aryl, —C(O)-heterocycle group, in particular a group represented by one of formulae (III) or (IV)

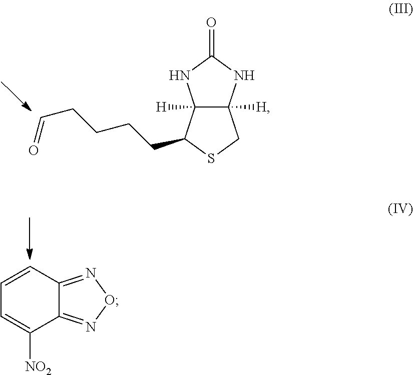

R₆ can represent a hydrogen atom or a halogen atom or a hydroxyl group, preferentially a hydrogen atom;
R₇ can represent a hydrogen atom, or an —OR^{a} group, R^{a} being as defined previously, preferentially a hydrogen atom;
   it being understood that
If X and Z together with the carbon to which they are attached represent a ketone (═O) group, an (═N—OH) oxime group or an alkyl (═N—O-alkyl) oxime group the alkyl of which group can be C₁-C₆ then
R₁ can represent a hydrogen atom or a —CH₃, —CH₂—CN, —CH₂—OR^{a}, —CH₂—SR^{a}, —CH₂—SeR^{a}, —C(O)—R^{a}, —C(O)OR^{a}, —O—C(O)NR^{a}R^{b}, —C(O)NR^{a}R^{b} group, R^{a} and R^{b} being as defined previously,
A can represent
   a hydrogen atom, or
   a C₁-C₈ alkyl group, or
   a C₂-C₈ alkenyl, or
   a C₂-C₈ alkynyl, or
   a C₃-C₆ cycloalkyl, or
   an aryl, or
   a heterocycle, or
   a halogen atom or
   a —(CH₂)ₙ—CN, —(CH₂)ₙ—CF₃, —(CH₂)ₙ—NO₂, —(CH₂)ₙ—OR^{a}, —(CH₂)ₙ—C(Me)₂OR^{a}, —(CH₂)ₙ—CHMeOR^{a}, —(CH₂)ₙ—SR^{a}, —(CH₂)ₙ—SO₂R^{a}, —CH₂—SeR^{a}, —(CH₂)ₙ—NR^{a}R^{b}, —C(O)R^{a}, —OC(O)NR^{a}R^{b}, —C(O)OR^{a}, —C(O)NR^{a}R^{b} group in which
      (i) R^{a} and R^{b}, are as defined previously and
      (ii) n can represent an equal integer which can have any one of the values from 0 to 4, or also
   a group corresponding to formula (V):

R^{c}-Q-(CH₂)ₘ (V)
in which
   (i) m can represent an integer which can have any one of the values from 1 to 8; and
   (ii) Q can represent an oxygen atom or an —NR^{a} group in which R^{a} is as defined previously and
   (iii) R^{c} can represent
      a. a hydrogen atom or
      b. a C₁-C₆alkyl,
      c. an aryl,
      d. a heteroaryl,
      e. a heterocycle,
      f. an alkyl-C(O)—, the alkyl of which can be C₁-C₆,
      g. an aryl-C(O)—,
      h. a heteroaryl-C(O)—,
      i. a heterocycle-C(O)—,
      j. a group represented by one of formulae (III) or (IV)

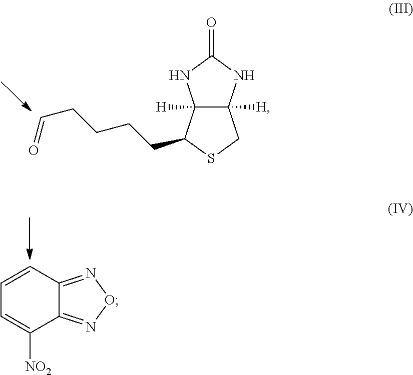

or
k. an —O—C(O)— group or
l. an —NR^{a}—C(O)— group in which R^{a} is as defined previously
If A with X together with the carbons to which they are attached represent a chain of formula (VI)

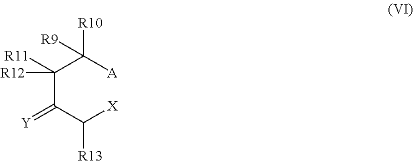

in which
Y can represent a ketone (═O) group, an (═N—OH) oxime group or an alkyl (═N—O-alkyl) oxime group the alkyl group of which can be C₁-C₆;
R₉ can represent a hydrogen atom, a C₁-C₆ alkyl group, a halogen atom; or
R₉ and R₁₁ together can form an additional carbon-carbon bond between the carbon atoms to which they are attached, or a C₃-C₆ cycloalkyl group;
R₁₀ can represent a hydrogen atom, a halogen atom or an —OR^{a}, —SR^{a}, —CN, —NR^{a}R^{b} group, —R^{a} and —R^{b} being as defined previously;
R₁₁ can represent a hydrogen atom, or a C₁-C₆ alkyl, C₃-C₆ cycloalkyl, aryl group or a halogen atom;
R₁₂ can represent a hydrogen atom, or a C₁-C₆ alkyl group or a halogen atom or a —CN, —OR^{a}, —SR^{a}, —SER^{a}, —C(O)—R^{a}, —C(O)OR^{a}, —NR^{a}R^{b}, —OC(O)NR^{a}R^{b} group, —R^{a} and —R^{b} being as defined previously; or
R₁₁ and R₁₂ together with the carbon to which they are attached, can form a C₃-C₆ cycloakyl group;
R₁₃ can represent a hydrogen atom or a C₁-C₆ alkyl group, or a halogen atom or a —CN, —CF₃, —NO₂, —OR^{a}, —SR^{a}, —SO₂R^{a}, —NR^{a}R^{b}, —C(O)—R^{a}, —OC(O)R^{a}, —OC(O)NR^{a}R^{b}, —C(O)OR^{a}, —C(O)NR^{a}R^{b} group or together with Z a carbon-carbon bond
then
Z can represent a hydrogen atom, a hydroxyl group, a hydroxyamino group or together with R₁₃ a carbon-carbon bond and
R₁ can represent a hydrogen atom or a —CH₃, —CH₂—CN, —CH₂—SR^{a}, —CH₂—SeR^{a} group or also a group corresponding to the following formula (VII) or (VIII):

—CH₂—W—R^{b} (VII) or

—C(O)—W—R^{b} (VIII)
in which
   W can represent an oxygen atom or an —NR^{a} group in which R^{a} is as defined previously or, a spacer arm constituted by an optionally substituted, linear or branched hydrocarbon chain comprising 2 to 20 carbon atoms and comprising moreover at least one heteroatom;
   R^{c} being as defined previously;
as well as:
   its SYN, ANTI geometrical isomers, when they exist,
   its optical isomers (enantiomers, diastereoisomers), when they exist,
   its addition salts with a pharmaceutically acceptable acid or base,
   its hydrates and its solvates,
   its prodrugs,
   or one of its esters.
      As a person skilled in the art understands, a certain number of compounds of formula (I) which comprise one or more hydroxyl groups, can be esterified. These esters as well as their addition salts with pharmaceutically acceptable acids are not generally directly active in themselves but constitute prodrugs for the corresponding hydroxylated analogues. These esters, which are metabolized in the human organism, lead to active compounds. These esters are also the subject of the present invention. The esters introducing chemical functionalities such as sulphates, phosphates, acids and basic chains which increase aqueous solubility and bioavailabilty can be mentioned. The esters of compounds bearing a basic function such as the analogues of dialkylglycine with alkyls with 1 to 4 carbon atoms and quite particularly dimethylglycine and diethylglycine and also methylpiperazine are preferred. The esters of fatty acids or the esters of a polyethyleneglycol chain which increase the affinity for the lipophilic phases can be mentioned. Saturated fatty acid chains with 3 to 18 carbons are preferred.

Moreover according to the present text,
the term “Cₓ—C_{y} alkyl refers to a linear or branched hydrocarbon radical, comprising x to y carbon atoms. Thus by way of example, the invention according to the cases listed covers linear or branched hydrocarbon radicals, such as methyl, ethyl, propyl, isopropyl, butyl, isobutyl, tert-butyl, pentyl, neopentyl, n-hexyl, n-heptyl, n-octyl, n-nonyl, n-decyl, n-undecyl, n-dodecyl. The C₁-C₆ alkyl groups are preferred. The alkyl groups can optionally be substituted by an aryl group as defined hereafter, in which case an arylalkyl group is mentioned. Examples of arylalkyl groups are in particular benzyl and phenethyl. Optionally, the alkyl groups can be substituted one or more times by one or more identical or different substituents, chosen independently from a halogen atom or a —CN, —CF₃, —C(O)—R^{a}, —C(O)OR^{a}, —C(O)NR^{a}R^{b}, —O—C(O)NR^{a}R^{b}, —NR^{a}R^{b}, —OR^{a}, —SR^{b} group, the R^{a} and R^{b} groups can be as described previously.
The term “Cₓ—C_{y} alkenyl” refers to a linear or branched or cyclic hydrocarbon radical, comprising one or more double bonds, having x to y carbon atoms. For example, the ethenyl, 1-propenyl, 2-propenyl, 1-butenyl, 2-butenyl, 1-pentenyl, 2-pentenyl, 3-methyl-3-butenyl, 1-hexenyl, 1-heptenyl, 1-octenyl radical can be mentioned. Optionally, the alkenyl groups can be substituted one or more times by one or more identical or different substituents chosen independently from a halogen atom or a —CN, —CF₃, —C(O)—R^{a}, —C(O)OR^{a}, —C(O)NR^{a}R^{b}, —O—C(O)NR^{a}R^{b}, —NR^{a}R^{b}, —OR^{a}, —SR^{b} group, the R^{a}, R^{b} groups can be as described previously;
the term “Cₓ—C_{y} cycloalkyl” refers to a saturated or partially unsaturated cyclic hydrocarbon radical, having x to y carbon atoms. The cycloalkyl groups include in particular the substituents cyclopropyl, cyclobutyl, cyclopentyl, cyclopentenyl, cyclohexyl, cyclohexenyl.
   Optionally, the cycloalkyl groups can be substituted one or more times by one or more identical or different substituents chosen independently from a halogen atom or a —CN, —CF₃, —C(O)—R^{a}, —C(O)OR^{a}, —C(O)NR^{a}R^{b}, —O—C(O)NR^{a}R^{b}, —NR^{a}R^{b}, —OR^{a}, —SR^{a} group, the R^{a}, R^{b} group can be as described previously;
the term “Cₓ—C_{y} alkynyl” refers to a linear, branched hydrocarbon radical comprising at least one triple bond, having x to y carbon atoms. The alkynyl groups include in particular the ethynyl, 1-propynyl, 2-propynyl, 1-butynyl, 2-butynyl, 1-pentynyl, 2-pentynyl, 1-heptynyl, 2-heptynyl, 1-octynyl, 2-octynyl substituents. Optionally, the alkynyl groups can be substituted one or more times by one or more identical or different substituents chosen independently from a halogen atom or a —CN, —CF₃, —C(O)—R^{a}, —C(O)OR^{a}, —C(O)NR^{a}R^{b}, —O—C(O)NR^{a}R^{b}, —NR^{a}R^{b}, —OR^{a}, —SR^{a} group, the R^{a}, R^{b} groups can be as described previously;
the term “Cₓ−C_{y} aryl” refers to an aromatic hydrocarbon radical, having x to y carbon atoms. Preferentially according to the invention the aromatic hydrocarbon radicals having 6 carbon atoms are preferred. The aryl groups include in particular the phenyl, naphthyl and bi-phenyl radicals. Optionally, the aryl groups can be substituted one or more times by one or more identical or different substituents chosen independently from a halogen atom or an alkyl, —CN, —CF₃, —N₃, —NO₂, —C(O)OR^{a}, —C(O)—R^{a}, —C(O)NR^{a}R^{b}, —O—C(O)NR^{a}R^{b}, —NR^{a}R^{b}, —OR^{a}, —SR^{a} group, the R^{a}, R^{b} groups can be as described previously;
the term “Cₓ—C_{y} heterocycle” refers to a mono- or poly-cyclic, saturated, unsaturated or aromatic, optionally substituted radical and can comprise x to y carbon atoms and comprising one or more heteroatoms. Preferably, the heteroatoms are chosen from oxygen, sulphur and nitrogen. Examples of heterocycles are the furyl, thienyl, pyrrole, imidazole, isothiazole, thiazole, isoxazole, oxazole, pyridine, pyrazine, pyrimidine, pyridazine, indole, isoindole, indazole, quinoline, isoquinoline, phthalazine, quinazoline, pyrrolidine, imidazolidine, pyrrazolidine, piperidine, piperazine, morpholine, thiazolidine or phthalimide, benzimidazole radicals. Optionally, the heterocyle groups can be substituted one or more times by one or more identical or different substituents chosen independently from a halogen atom or an alkyl, —CN, —CF₃, —N₃, —NO₂, —C(O)OR^{a}, —C(O)—R^{a}, —C(O)NR^{a}R^{b}, —O—C(O)NR^{a}R^{b}, —NR^{a}R^{b}, —OR^{a}, —SR^{a} group, the R^{a}, R^{b} groups can be as described previously;
the term “halogen” refers to a chlorine, bromine, fluorine and iodine atom. Preferentially according to the invention, the halogen is a fluorine atom.

The compounds of formula (I) are known and are particularly described in the international applications WO 2006/027454 of 13 Mar. 2006, WO 2007/101925 of 13 Sep. 2007, WO 2009/044011 of 9 Sep. 2009, WO 2010/076418 of 8 Jul. 2010, WO 2004/082581 of 30 Sep. 2004, WO 2007/080270 of 19 Jul. 2007, WO2007/118967 of 25 Oct. 2007, WO 008/142237 of 27 Nov. 2008, WO 2009/044010 of 9 Apr. 2009, WO 2009/092892 of 30 Jul. 2009 and WO 2010/012904 of 4 Feb. 2010.

It is well known (see the international applications cited previously) that the cholesterol derivatives, such as particularly for example those of formula (I), particularly 3-hydroxy-3,5-seco-4-norcholestan-5-one oxime and cholest-4-en-3-one oxime can be used as medicaments and particularly have remarkable cytoprotective properties, particularly neuroprotective, cardioprotective and/or hepatoprotective.

But these compounds are very lipophilic and very insoluble in aqueous medium compatible with an administration for example a parenteral administration, which makes their introduction into compositions, particularly pharmaceutical compositions, particularly difficult or even impossible. It is therefore difficult to obtain such compositions that are chemically and physically stable.

In the prior art, the development of compositions comprising active ingredients having a low aqueous solubility, traditionally focused on the use of surfactants which allow the formation of emulsions, colloids such as micelles or liposomes which solubilize the medicament and increase its solubility in aqueous medium. Nevertheless, these emulsions and in particular the micellar suspensions are not physically or even chemically stable. For example if the composition comes into contact with blood or plasma, the solubilizing system can thus lose these properties, retaining only the active ingredient.

Moreover, the use of surfactant (cremophore, tween, etc.) and the formation of particulate structures give rise to the activation of the complement system of higher organisms and the initiation of reactogenicity reactions which can be fatal.

These problems of solubility and reactogenicity become all the more difficult when it is sought to obtain a composition having a high concentration of active ingredient, allowing inter alia the administration of a volume of composition as small as possible or in a shorter time to be envisaged.

The invention aims inter alia to overcome these problems and difficulties.

The applicant has, surprisingly, discovered that the cholesterol derivatives, particularly those described in the international applications cited previously, advantageously those corresponding to formula (I), have a very good affinity for liposomes, which moreover allows the preparation of compositions, particularly pharmaceutical compositions, which are physically and chemically stable for several months and concentrated with active ingredient. In addition these compositions when in contact with a biological medium such as blood are chemically stable and the active ingredient retains all its properties.

Moreover, these compositions once administered surprisingly have a very low reactogenicity.

Moreover, the liposomes, according to the invention, have a prolonged remanence in biological liquids, particularly in blood.

The invention resides in the fact that the liposomes according to the invention allow a much more significant solubilization of the compounds of formula (I) while having a very low reactogenicity. Thus the compounds of formula (I), when included in the liposomes according to the invention, can be formulated in pharmaceutical compositions in contact with aqueous media much more easily than if they were presented in another form.

The liposomes consist of at least one lipid bilayer membrane surrounding an aqueous internal compartment. They are known as an effective system of formulations for transporting therapeutic agents, drugs, or active ingredients within the aqueous space present inside the vesicle (agents soluble in aqueous medium) or included in the lipid bilayer (agents insoluble in aqueous medium). They can be characterized by the type of membrane and their size. The unilamellar vesicles are constituted by a single bilayer membrane. The multilamellar vesicles (MLV: multilamellar vesicle) have at least two lipid bilayer membranes defining several aqueous closed compartments. The membranes are organized in a concentric fashion so that the different membranes are separated by an aqueous compartment.

The small unilamellar vesicles (SUV: small unilamellar vesicle) can have a diameter generally comprised between 20 and 100 nm. The large unilamellar vesicles (LUV: large unilamellar vesicle) and the multilamellar vesicles (MLV: multilamellar vesicle) can have a diameter generally greater than 100 nm.

As regards the liposomes, it is possible to refer to the work of Gregory Gregoriadis (Liposome Technology: Liposome Preparation And Related Techniques, 3rd edition, 2007) for their description, manufacture and applications.

But one of the major drawbacks with the development of the liposomal formulations, on the experimental scale for the pharmaceutical market, is their chemical and physical instability and their relative stability during production and storage.

Therefore, since Bangham in 1965 (Bangham A D., Standish M M., Watkins J C. (1965). J. Mol. Biol. 13, p. 238-252) who was the first to manufacture liposomes, numerous improvements have been made in particular for increasing their steric stabilization and for increasing their residence time in the vascular system which in particular makes it possible to limit injections or even to envisage targeted therapies or delayed forms.

Among these improvements “PEGylation” can be mentioned as an example which consists of the introduction of PEG (Poly Ethylene Glycol) chains often grafted on phospholipids or cholesterol.

The introduction of sterols such as cholesterol into the lipid bilayer allows the stabilization of the liposomes to be improved.

The U.S. Pat. No. 6,143,321 describes liposomes the active ingredient of which is entrapped or adsorbed in the lipid bilayer using a surfactant.

An improvement in the physical and chemical stability of the liposomes according to the invention represents one of the great advantages of the invention as it makes it possible to envisage the preparation of low-volume compositions comprising a large quantity of active ingredient, which would make it possible to envisage, particularly in the case of pharmaceutical compositions, delivering to patients only small doses or even a single dose of medicaments, which can make it possible to limit or even eliminate hypersensibilization (reactogenicity) problems that could cause the administration of several doses of medicament solubilized using liposomes.

In addition, as the active ingredient can be comprised in the liposomes according to the invention, it is sufficient in itself to confer an improved stability on the liposomes without adding extra sterol.

Another major drawback in the pharmaceutical development of liposomal formulation in particular by parenteral route is the increased risk of reactogenicity linked to the activation of the complement (Szebeni J., Toxicology, 2005, 216, 106). These reactions are even more frequent or intense when the administration of the solution is carried out at a high rate such as with a bolus. A weakly reactogenic pharmaceutical composition would allow a parenteral administration by bolus and not by infusion of a massive quantity of active ingredient. Moreover this formulation would allow a rapid administration by bolus and preferentially manually.

By “weakly reactogenic pharmaceutical composition” is meant in the present text that the group having received the composition induces a level of reactogenicity similar to that induced in the control group of the trial having only received the buffer of this composition.

An administration by bolus corresponds to the administration of a quantity necessary in order to obtain the expected concentration in the blood, the lymph, the cerebrospinal fluid or any targeted biological site for therapeutic or diagnostic reasons in a short period of time comprised between 1 second and 10 minutes, preferentially in less than 5 minutes, very preferentially in less than 2 minutes.

A weakly reactogenic composition increases the level of the complement by a maximum of three times with respect to the basal level measured in the serum of the individual. The level of terminal C complex (SC5b-9) can be measured by an immunological test kit such as that from the Quibel Corporation “SC5b-9 plus Elisa” Kits.

It is one of the purposes of the invention to provide weakly reactogenic liposomes, which can be used alone or in pharmaceutical compositions.

Thus the properties of the liposomes according to the invention make it possible to prepare compositions, preferentially pharmaceutical compositions, having the following advantages:
physical and chemical stability for at least 12 months in storage phase at 25° C.;
possible use for an administration by bolus by parenteral route;
very low reactogenicity;
process which can be adapted to industrial production;
integration with other active ingredients, with the possible option of a massive administration by bolus.

Thus a first subject of the invention is liposomes comprising at least one of the compounds corresponding to formula (I) or a mixture of compounds corresponding to formula (I).

A subject of the invention is also a composition, particularly a pharmaceutical composition comprising liposomes comprising at least one of the compounds corresponding to formula (I) or a mixture of compounds corresponding to formula (I).

A person skilled in the art has a good knowledge of liposomes and the techniques not only for preparing them but also for introducing an active agent into them.

The invention relates to any known type of liposome providing it is used to encapsulate an active agent, particularly a compound of formula (I).

According to a particular form of the invention, the final liposomal solution can comprise at least:
a compound of formula (I);
a phospholipid or a mixture of phospholipids;
an agent that stabilizes the pH;
and optionally a cryoprotective agent.

Advantageously the compound of formula (I) comprised in the liposomes according to the invention can be chosen from
cholest-4-en-3-one oxime,
cholest-4,24-dien-3-one oxime,
cholestan-3-one oxime,
3-hydroxy-3,5-seco-4-norcholestan-5-one oxime,
1,4-cholestadien-3-one oxime,
2-methyl-cholest-4-en-3-one oxime,
2α-fluoro-cholest-4-en-3-one oxime,
4-methoxy-cholest-4-en-3-one oxime,
4-fluoro-cholest-4-en-3-one oxime,
6β-fluoro-cholest-4-en-3-one oxime,
19-hydroxy-cholest-4-en-3-one oxime,
19-biotinyloxy-cholest-4-en-3-one oxime,
25-((N-(+)-biotinoyl-N-methyl)amino)-27-norcholest-4-en-3-one oxime,
25-[methyl(7-nitro-2.1,3-benzoxadiazol-4-yl)amino]-27-norcholest-4-en-3-one oxime,
2,2-difluoro-cholest-4-en-3-one oxime,
2,6-difluoro-cholest-4-en-3-one oxime,
cholest-4-en-3,6-dione 3-oxime,
cholest-4,21-dien-3,6-dione 3-oxime,
24-ethyl-cholest-4-en-3,6-dione 3-oxime,
24-ethyl-cholest-4,21-dien-3,6-dione 3-oxime,
24-methyl-cholest-4,21-dien-3,6-dione 3-oxime,
3-[methyl(7-nitro-2.1,3-benzoxadiazol-4-yl)amino]-3,5-seco-4-norcholestan-5-one oxime,
2-hydroxy-2,5-seco-3,4-dinor-cholestan-5-one oxime,
3-[(N-(+)-biotinoyl-N-methyl)amino]-3,5-seco-4-norcholestan-5-one oxime,
25-fluoro-3-hydroxy-3,5-seco-4-norcholestan-5-one oxime,
3,5-seco-4-norcholestan-5-one oxime,
3-hydroxy-3,5-seco-4-norcholest-24-en-5-one oxime,
3-hydroxy-3,5-seco-3-trifluoromethyl-4-norcholestan-5-one oxime,
24β-ethyl-3-hydroxy-3,5-seco-4-norcholest-22-en-5-one oxime,
3-hydroxy-3-methyl-3,5-seco-4-norcholestan-5-one oxime,
3,3-dimethyl-3-hydroxy-3,5-seco-4-norcholestan-5-one oxime,
3-hydroxy-3,5-seco-4-norcholestan-5-one oxime,
cholest-4-en-3,6-dione dioxime,
cholest-4,24-dien-3,6-dione dioxime,
24-methyl-cholest-4,21-dien-3,6-dione dioxime,
24-ethyl-cholest-4-en-3,6-dione dioxime,
24-ethyl-cholest-4,21-dien-3,6-dione dioxime,
cholest-4-en-3-one,
cholest-4,24-dien-3-one,
3-hydroxy-3,5-seco-4-norcholestan-5-one,
3-hydroxy-3-methyl-3,5-seco-4-norcholestan-5-one,
3,3-dimethyl-3-hydroxy-3,5-seco-4-norcholestan-5-one,
3-hydroxy-3,5-seco-4-norcholestan-5-one methyloxime.

Very preferentially the compound of formula (I) comprised in the liposomes according to the invention can be chosen from cholest-4-en-3-one oxime, cholestan-3-one oxime, cholest-1,4-dien-3-one oxime, cholest-4,24-dien-3-one oxime, 3-hydroxy-3,5-seco-4-norcholestan-5-one oxime, 4-fluoro-cholest-4-en-3-one oxime, 3-hydroxy-3-methyl-3,5-seco-4-norcholestan-5-one oxime, 3,3-methyl-3-hydroxy-3,5-seco-4-norcholestan-5-one oxime, very preferentially cholest-4-en-3-one oxime and 3-hydroxy-3,5-seco-4-norcholestan-5-one oxime.

Of course, the invention also relates to the liposomes for which a mixture of at least two compounds of formula (I) can be used for their constitution.

According to the invention the compound of formula (I) can be present in the liposome at physiologically effective doses. According to the invention, the compound of formula (I) can be present, in the final liposomal solution, in a quantity ranging from 0.1 to 200 mg/mL, preferentially below 1 mg/mL, also more preferentially below 5 mg/mL, very preferentially below 10 mg/mL).

By “final liposomal solution” is meant the solution of liposomes obtained after implementation of the process making it possible to obtain the liposomes according to the invention.

According to the invention, the phospholipids used can be phospholipids of natural, vegetable or animal, or synthetic origin, yet more advantageously natural phospholipids. Said phospholipids can be chosen from phosphoacylglycerols (better known under the name of glycerophospholipids), inositophosphatides, phosphosphingolipids and phosphonosphingolipids or also phosphosaccharolipids.

Preferentially the phospholipids can be chosen from phosphatidylcholine, lysophosphatidylcholine, phosphatidylethanolamine, linoleylpalmitoylphosphatidylcholine, palmitoyloleoylphosphatidylcholine, oleoylpalmitoylphophatidylcholine, DHAstearoylphosphatidyl choline, DHA-rich phosphatidylcholine of avian origin, phosphatidylinositol, DHA-phosphatidylethanolamine, phosphatidylserine, sphingomyelin, a mixture of phospholipids of avian origin close to the composition of human milk, a mixture of phospholipids of soya origin close to the composition of human milk, lysophosphatidic palmitic or oleic acids, egg lysophosphatidylcholine containing palmitic and stearic acids at more than 90%, soya lysophosphatidylcholine, lysophosphatidylinositol, lysophosphatidylethanolamine, lysophosphatidylserine, a mixture of egg phospholipids containing phosphatidylcholine, phosphatidylethanolamine, phosphatidylserine, phosphatidylinositol and sphingomyelin, 1,2-dioleolyl-sn-glycero-3-phosphocholine (DOPC), 1,2-Dimyristoyl-sn-glycero-3-phosphorylcholine (DM PC), 1,2-dimyristoylphosphatidylglycerol (DMPG), egg L-α-phosphatidylcholine, soya L-α-phosphatidylcholine, 1,2-distearoyl-sn-glycero-3-phosphoethanolamine-N-[methoxy(polyethylene glycol)-2000] (DSPE-PEG).

Preferably, according to the invention phospholipids of natural origin are used and particularly egg or soya phospholipids, preferably egg phospholipids.

Of course, the invention also relates to liposomes for which a mixture of at least two phospholipids can be used for their constitution.

According to the invention the phospholipids can be present in the final liposomal solution, in a quantity ranging from 10 to 300 mg/mL, preferentially between 20 and 200 mg/mL, very preferentially between 50 and 150 mg/mL).

A person skilled in the art will without difficulty establish the ratio of the concentration of the compound of formula (I) to that of the phospholipid to be used in order to obtain the liposomes according to the invention, particularly with reference to Gregory Gregoriadis (Liposome Technology: Liposome Preparation And Related Techniques, 3rd edition, 2007).

By way of example, but without being limitative, it can be indicated that the ratio of the concentration of the compound of formula (I) to that of the phospholipid can be comprised in the final liposomal solution between 1/100 and 70/100, preferentially between 10/100 and 50/100.

According to the invention, the pH stabilizing agent can be a phosphate, benzoate, citrate, glutamate, lactate, ascorbate, tartrate, succinate, adipate, glycinate, malate, triethanolaminate, diethanolaminate, tromethaminate buffer.

Preferably, according to the invention a phosphate buffer is used.

According to the invention the pH stabilizing agent can allow the pH of the liposomal formulation to vary between 3 and 11, preferentially between 4 and 9. A person skilled in the art will without difficulty adjust the quantity of stabilizing agent to be introduced as a function on the one hand of the final pH of the liposomal formulation that it is intended to obtain and as a function of the buffer chosen and its physical properties.

Similarly according to the invention, the cryoprotective agent can be for example glycerol, sucrose, dextrose, trehalose, glucose, maltose, mannose, lactose, mannitol, sorbitol, glycine, polyvinylpyrrolidone (PVP), polyvinylalcohol (PVA), gelatin, alanine, lysine, polyethylene glycol, dextran, aerosil, fructose, hydroxypropyl-β-cyclodextrin. Preferentially according to the invention, glycerol, sucrose, dextrose are used.

According to the invention the cryoprotective agent can be present in the liposomal formulation in a quantity ranging from 0.01 to 30% in solution, preferentially between 0.1 and 20%, very preferentially between 1 and 10%.

According to the invention the compound of formula (I) can be combined with other active therapeutic agents. Thus, it is possible to combine this active ingredient with another complementary or secondary ingredient incorporated either in said lipid layer of the liposome, or in the aqueous phase depending on their solubility.

The liposome according to the present invention can be used alone or in a composition in animals or humans, particularly mammals, more precisely in humans. They can be for cosmetic, pharmaceutical or veterinary use.

Thus a subject of the invention is also a composition comprising at least one liposome comprising at least one compound of formula (I).

It is known that the composition of the invention can also contain the usual adjuvants in the fields considered depending on the administration method, such as for example preservatives, antioxidants, pigments and colouring materials, thickeners, fragrances, sweeteners, agents stabilizing the active particles.

The quantities of these different adjuvants are those used in a standard fashion in the fields considered, and are for example from 0.0001% to 10% of the total weight of the composition. These adjuvants are introduced into the aqueous or lipophilic phase.

According to the invention, the liposome or the composition comprising at least one liposome, can be administered by enteral, parenteral or topical route, preferentially by parenteral route. By parenteral route, the administration can be carried out by intravenous or intraarterial or intralymphatic, direct (syringe) or indirect route (by a perfusion or an angioplasty catheter).

In the case of a vein this can be superficial, usually in the arm (peripheral venous route) or deep (central venous route), most often in the neck (jugular vein) or under the clavicle (sub-clavian vein), by sub-cutaneous route, under the skin, frequently in the stomach or thighs, by intradermal route, directly into the dermis, by intramuscular route, directly into a muscle or also by pulmonary route by inhalation. Preferentially, the administration can be carried out by intravenous route.

A subject of the invention is also the use of the compounds of formula (I) for preparing liposomes, advantageously stable liposomes.

A subject of the invention is also the use of the liposomes according to the invention for preparing a composition, advantageously a weakly reactogenic, cosmetic, pharmaceutical, or veterinary composition.

Moreover, a subject of the invention is the use of the liposomes according to the invention for the transport of active ingredients other than the compounds of formula (I).

Other features and properties of the invention will become apparent on reading the following examples which illustrate the invention without thereby limiting it.

Abbreviations used:
EPC: Egg L-α-phosphatidylcholine
SPC: Soya L-α-phosphatidylcholine
DOPC: 1,2-dioleoyl-sn-glycero-3-phosphocholine
DMPG: 1,2-dimyristoylphosphatidylglycerol
DSPE-PEG: 1,2-distearoyl-sn-glycero-3-phosphoethanolamine-N-[methoxy(polyethylene glycol)-2000]
PBS: Phosphate buffer saline
Eth: Ethanol
terBut: tert-Butanol

List of the compounds of formula (I) tested:

| N^{o} | Chemical name |
|---|---|
| 1 | 3-hydroxy-3,5-seco-4-norcholestan-5-one oxime |
| 2 | 3,3-dimethyl-3-hydroxy-3,5-seco-4-norcholestan-5-one oxime |
| 3 | cholest-4-en-3-one oxime |
| 4 | cholest-4,24-dien-3-one oxime |
| 5 | 4-fluoro-cholest-4-en-3-one oxime |
| 6 | 3,5-seco-4-norcholestan-5-one oxime |
| 7 | cholest-4-en-3-one |
| 8 | 3-hydroxy-3,5-seco-4-norcholestan-5-one |
| 9 | cholest-4,24-dien-3-one |

### EXAMPLE 1

### Comparison of the Solubility of the Derivatives of Formula (I)

The maximum solubility of the compounds of formula (I) was tested in water and different solvents:

| | Compound N^{o} | | | |
|---|---|---|---|---|
| Solvent | 1 | 3 | 4 | 6 |
| Water | 0.001 | 0.0001 | 0.0001 | 0.0001 |
| Soya oil | 6 | 30 | | |
| (2-hydroxypropyl)-beta- | 0.7 | | | |
| cyclodextrin, 30% PBS buffer | | | | |
| Intralipid ®10 | 1 | | 0.34 | 0.4 |
| Intralipid ®10/SolutolHS15 ®/ | 5 | | | |
| ethanol 92/3/5 | | | | |
| Liposome based on egg | 30 | 12.4 | 14.5 | 8.2 |
| phospholipids | | | | |

Concentration in mg/mL
Intralipid ®: injectable lipid emulsion, generally used in parenteral nutrition, sold by the laboratory Fresenius Kabi France
Solutol HS15 ®: non ionic solubilizing agent sold by the company BASF.
CONCLUSION: The liposomal formulation allows a solubility of the compounds of formula (I) in an aqueous medium of greater than or equal to 8 mg/mL and at a concentration equal to or greater than a medium which is highly lipophilic but not miscible in an aqueous biological medium.

### EXAMPLE 2

### Preparation of Suspensions of Liposomes Containing the Derivatives of Formula (I)

A solution of lipids at 250 mg/mL is prepared by dissolving defined quantities of EPC and the compound of formula (I) in tert-butanol or a mixture of tert-butanol and absolute ethanol (v/v). This lipid solution is mixed at a certain temperature with aqueous buffer in order to obtain a final concentration of solvent of 15 to 17% and of product of formula (I) of 25 or 50 mg/mL.

The large multilamellar vesicles are then extruded at a fixed temperature through 3 polycarbonate filters by 10 consecutive passes. The non encapsulated compound of formula (I) is removed on the filters.

The solvent is eliminated by filtration on membrane by washing with buffer in order to achieve a percentage of final solvent of the order of 0.4%.

| A | B | C | D | E | F | G | H |
|---|---|---|---|---|---|---|---|
| 1 | Eth/terBut | 25 | 25 | 50/200 | 8.1 | 15-20 | 81.4 |
| 2 | Eth/terBut | 25 | 25 | 50/200 | 9.2 | 15-20 | 79.5 |
| 3 | Eth/terBut | 40 | 45 | 50/200 | 12.4 | 15-20 | 88.9 |
| 4 | Eth/terBut | 80 | 45 | 50/200 | 14.5 | 15-20 | 80.7 |
| 5 | terBut | 80 | 60 | 25/100 | 3.6 | 30-45 | 79.1 |
| 6 | Eth/terBut | 80 | 55 | 50/200 | 8.2 | 25-35 | 84.7 |
| 7 | Eth/terBut | 25 | 25 | 50/200 | 12.1 | 15-20 | |
| 8 | Eth/terBut | 25 | 25 | 50/200 | 11.4 | 15-20 | |
| 9 | Eth/terBut | 25 | 25 | 50/200 | 10.1 | 15-20 | |

A: Compound
B: Solvent(s)(v/v)
C: Solubilization temperature in ° C.
D: Extrusion temperature in ° C.
E: Initial concentration in mg/mL of buffer [Compound/EPC]
F: Concentration of compound in mg/mL in the liposomes
G: Extrusion pressure (Bars)
H: Particle size (nm)

CONCLUSION: The solutions of liposomes with the compounds were produced with concentrations between 3 and 15 mg/mL. The size of the vesicles is very homogenous from one compound to the other, of the order of 80 nm corresponding to small unilamellar vesicles.

### EXAMPLE 3

### Preparation of Suspensions of Liposomes Containing 3-hydroxy-3,5-seco-4-norcholestan-5-one oxime (Compound 1)

A solution of lipids at 250 mg/mL is prepared by dissolving defined quantities of phospholipids and compound 1 in a mixture of tert-butanol and absolute ethanol (v/v). This lipid solution is mixed at ambient temperature with aqueous buffer in order to obtain a final concentration of solvent of 20% and of total lipids of 50 mg/mL.

The large multilamellar vesicles are then extruded at ambient temperature through 3 to 5 polycarbonate filters. 5 to 10 passes are necessary to generate the small unilamellar vesicles with a size between 70 and 90 nm.

The solvent and non encapsulated 3-hydroxy-3,5-seco-4-norcholestan-5-one oxime are removed by diafiltration using 10 volumes of washing buffer. An ultrafiltration is then carried out in order to concentrate the formulation to the final concentration.

| A | B | C | D | E | F |
|---|---|---|---|---|---|
| 1 | EPC | 10 mM Phosphate | 81.40 | 31.30 | 73.00 |
| | | 300 mM Sucrose | | | |
| 2 | DOPC | 10 mM Phosphate | 51.50 | 22.06 | 212.00 |
| | DSPE-PEG (96/4) | 300 mM Sucrose | | | |
| 3 | EPC | 10 mM Phosphate | 53.20 | 25.30 | 70.80 |
| | | 145 mM NaCl | | | |
| 4 | SPC-DMPG (96/4) | 10 mM Phosphate | 63.30 | 27.80 | 70.70 |
| | | 145 mM NaCl | | | |
| 5 | DOPC-DMPG (96/4) | 10 mM Phosphate | 71.70 | 29.30 | 95.70 |
| | | 145 mM NaCl | | | |
| 6 | EPC | 10 mM Phosphate | 63.40 | 26.20 | 57.40 |
| | | 300 mM sucrose | | | |
| 7 | EPC | 10 mM Phosphate | 46.30 | 21.88 | |
| | | 300 mM sucrose | | | |
| | | 145 mM NaCl | | | |
| 8 | EPC | 10 mM Phosphate | 46.30 | 22.00 | |
| | | 5% dextrose | | | |
| | | 145 mM NaCl | | | |
| 9 | EPC | 10 mM Phosphate | 46.30 | 22.00 | |
| | | 5% glycerol | | | |
| | | 145 mM NaCl | | | |
| 10 | EPC | 10 mM Phosphate | 66.50 | 27.70 | 69.20 |
| | | 5% glycerol | | | |
| 11 | EPC | 10 mM Phosphate | 76.7 | 19.6 | 67.0 |
| | | 5% glycerol | | | |

A: Composition (compound 1 + phospholipids of column B)
B: Phospholipids used for preparing the liposomes
C: Buffer
D: Concentration of phospholipids in mg/mL
E: Final concentration of compound 1 in mg/mL
F: Size of the vesicles in nm

CONCLUSION: The different liposomal compositions with concentrations around 20 to 35 mg/mL have small unilamellar vesicles of 50 to 100 nm, with the exception of composition 2. A low percentage of DSPE-PEG appears to form large unilamellar or multilamellar vesicles of 220 nm in the same final concentration range in compound 1.

### EXAMPLE 4

### In Vitro Evaluation of the Reactogenicity

The in vitro evaluation of the reactogenicity is carried out by measuring the production of SC5b-9 complex after incubation in human serum. The levels of SC5b-9 are measured with an ELISA kit according to the method described (QuidelCorporation; SC5b-9 More EIA kit, Ref. A029).

The solutions tested are diluted with buffer in order to achieve a concentration of compound of formula (I) of 20 mg/mL. 1 volume of liposomal solution prepared in Example 3 is added to 3 volumes of serum in order to achieve a tested final concentration of 5 mg/mL. The mixture is stirred vigorously and incubated for 45 minutes at 37° C. The reaction is stopped with the “stop” solution from the Elisa kit.

The levels of production of the SC5b-9 complex in the sera were measured with, as control:
the average of the concentrations obtained over all the sera alone (negative control) (C1),
the buffers (T1, T2)
a positive control (Zymosan-A®, polysaccharide of yeasts sold by SIGMA ALDRICH)
a reference compound (AmBisome®) (positive liposomal control).

Solution

| N^{o} | A | B | C | D |
|---|---|---|---|---|
| | | Serum | | 0.7 |
| | | Zymosan ® | | 170 |
| | | Ambisome ® | | 30.7 |
| T1 | | 10 mM Phosphate | | 2.3 |
| | | 300 mM Sucrose | | |
| T2 | | 10 mM Phosphate | | 5.3 |
| | | 145 mM NaCl | | |
| 1 | EPC | 10 mM Phosphate | 31.30 | 8.4 |
| | | 300 mM Sucrose | | |
| 2 | DOPC | 10 mM Phosphate | 22.06 | 9.7 |
| | DSPE-PEG | 300 mM Sucrose | | |
| | (96/4) | | | |
| 3 | EPC | 10 mM Phosphate | 25.30 | 7.8 |
| | | 145 mM NaCl | | |
| 4 | SPC-DMPG | 10 mM Phosphate | 27.80 | 7.6 |
| | (96/4) | 145 mM NaCl | | |
| 5 | DOPC- | 10 mM Phosphate | 29.30 | 7.9 |
| | DMPG (96/4) | 145 mM NaCl | | |
| 7 | EPC | 10 mM Phosphate | 21.88 | 11.7 |
| | | 300 mM sucrose | | |
| | | 145 mM NaCl | | |
| 8 | EPC | 10 mM Phosphate | 22.00 | 12.3 |
| | | 5% dextrose | | |
| | | 145 mM NaCl | | |
| 9 | EPC | 10 mM Phosphate | 22.00 | 5.8 |
| | | 5% glycerol | | |
| | | 145 mM NaCl | | |
| 10 | EPC | 10 mM Phosphate | 27.70 | 2.1 |
| | | 5% glycerol | | |

N^{o}: number of the solution tested.
A: Phospholipids
B: Buffer
C: Concentration of compound mg/mL
D: Concentration of SC5b-9 (μg/mL)

CONCLUSION: The level of SC5b-9 obtained in the serum with the positive liposomal control Ambisome® is 3 to 15 times greater than the levels obtained with the liposomal solutions prepared in Example 3. These induce the formation of SC5b-9 at low levels, similar to those obtained with the buffer or the serum alone. The liposomal solutions prepared are therefore weakly reactogenic.

### EXAMPLE 5

### Evaluation of the Reactogenicity of the Liposomal Formulation in Humans

A Phase 1 clinical study was carried out with the liposomal formulation No. 11 exemplified in Example 3 of 3-hydroxy-3,5-seco-4-norcholestan-5-one oxime. The formulation was administered to 54 healthy volunteers by intravenous route in several doses and flow rates according to the protocol filed with AFSSAPS and with the “Comite de protection des personnes” (Committee on the protection of individuals). This formulation showed a very good tolerance up to the maximum dose tested of 13 mg/kg at 10 mL/min and with a maximum flow rate of 35 mL/min for the dose of 10 mg/kg as well as a good demonstration of the product of formula (I).

### Conclusion:

No significant clinical sign relating to the activation of the complement was observed in humans in this study.

### EXAMPLE 6

### Evaluation of the Stability of the Liposomal Formulation

The solution of liposome No. 11 exemplified in Example 3 of 3-hydroxy-3,5-seco-4-norcholestan-5-one oxime was subjected to stability testing at +5 and +25° C. and analyzed after 3, 6, 12 months.

| Parameters | T0 | 3 months | 6 months | 12 months |
|---|---|---|---|---|
| Appearance | Translucent | Translucent | Translucent | Translucent |
| | solution | solution | solution | solution |
| Crystallization | Absence of | Absence of | Absence of | Absence of |
| | crystals | crystals | crystals | crystals |
| Size of the | 67.0 | 68.3 | 68.9 | 70.7 |
| vesicles (nm) | | | | |
| Concentration | 19.6 | 19.2 | 19.2 | 18.4 |
| in mg/mL | | | | |
| Impurities | 2.38 | 2.29 | 2.52 | 2.25 |
| (% of area) | | | | |

Conclusion:

The parameters measured are stable for at least a year at 25° C. The liposomal solution can be stored for at least 1 year at 25° C.

1. A liposome, comprising at least one compound of the following formula (I) 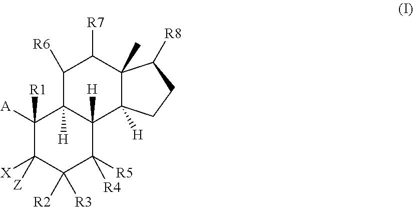
in which,
R₂ represents a hydrogen atom or a C₁-C₆ alkyl, C₃-C₆ cycloalkyl, C₂-C₆ alkenyl, C₂-C₆ alkynyl, aryl, a heterocycle group, or a halogen atom or a —CN, —CF₃, —NO₂, —OR^{a}, —SR^{a}, —SO₂R^{a}, —NR^{a}R^{b}, —C(O)—R^{a}, —OC(O)R^{a}, —OC(O)NR^{a}R^{b}, —C(O)OR^{a}, —C(O)NR^{a}R^{b} group, in which
(i) R^{a} and R^{b}, simultaneously or independently of one another, are chosen from a hydrogen atom or a C₁-C₆ alkyl group, a C₂-C₆ alkenyl group, a C₃-C₆ cycloalkyl group, an aryl group, a heterocyclic group or
(ii) R^{a} and R^{b} together form a linear or branched hydrocarbon chain, having 2 to 6 carbon atoms, optionally comprising one or more double bonds and/or optionally interrupted by one or more oxygen, sulphur or nitrogen atom(s), or
(iii) R^{a} and R^{b} together with the nitrogen to which they are attached form a C₃-C₆ heterocycle, said heterocycle comprising one or more double bonds and/or one or more oxygen, sulphur or nitrogen atom(s);
R₃ represents a hydrogen atom or a C₁-C₆ alkyl group,
or
R₂ and R₃ together with the carbon to which they are attached, represent an oxygen atom or a C₃-C₆ cycloalkyl group or an ═N—OH, ═CH—(C₁-C₆)alkyl, ═CH-aryl, ═CH—(C₃-C₆)cycloalkyl group,
R₄ represents a hydrogen atom or a C₁-C₆ alkyl group, or a hydroxyamino (—NH₂—OH) group; or
R₃ and R₄ together form an additional carbon-carbon bond between the carbon atoms to which they are attached, or a C₃-C₆ cycloalkyl group;
R₅ represents a hydrogen atom or a C₁-C₆ alkyl, C₃-C₆ cycloalkyl, C₂-C₆ alkenyl, C₂-C₆ alkynyl, aryl group, or a —CN, —OR^{a}, —SR^{a}, —SO₂R^{a}, —NR^{a}R^{b}, —C(O)—R^{a}, —OC(O)R^{a}, —OC(O)NR^{a}R^{b} group, R^{a} and R^{b} being as defined previously, or a hydroxyamino (—NH₂—OH) group;
R₈ represents a group chosen from
(i) a C₄-C₁₂ alkyl group or a C₄-C₁₂ alkenyl group, in particular a group chosen from 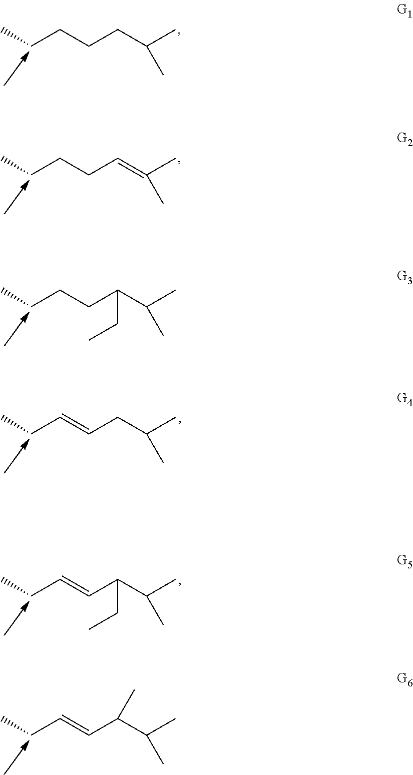
(ii) a group corresponding to formula (II) as follows:
R₁₄—Y—R₁₅ (II)
in which:
R₁₄ represents a C₄-C₁₂ alkyl group or a C₄-C₁₂ alkenyl group, in particular a C₅-C₁₀ alkyl group, preferentially the following G₇ group 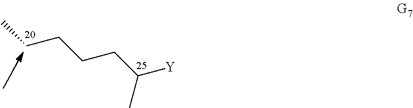
and
Y represents an oxygen atom or an —NR^{a} group with R^{a} being as described previously, and
R₁₅ represents a C₁-C₆ alkyl, C₃-C₆ cycloalkyl, aryl, heterocycle, —C(O)—(C₁-C₆)alkyl, —C(O)—(C₃-C₆)cycloalkyl, —C(O)-aryl, —C(O)-heterocycle group, in particular a group represented by one of formulae (III) or (IV) 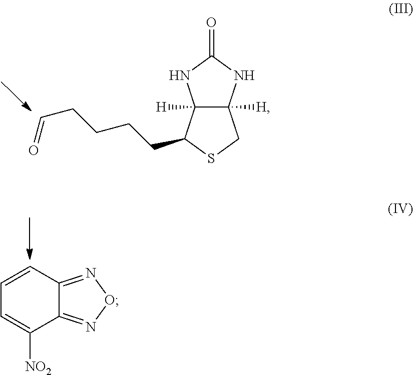
R₆ represents a hydrogen atom or a halogen atom or a hydroxyl group, preferentially a hydrogen atom;
R₇ represents a hydrogen atom, or an —OR^{a} group, R^{a} being as defined previously, preferentially a hydrogen atom;
it being understood that
If X and Z together with the carbon to which they are attached have a ketone (═O) group, an (═N—OH) oxime group or an alkyl (═N—O-alkyl) oxime group, the alkyl of which group is C₁-C₆, then
R₁ represents a hydrogen atom or a —CH₃, —CH₂—CN, —CH₂—OR^{a}, —CH₂—SR^{a}, —CH₂—SeR^{a}, —C(O)—R^{a}, —C(O)OR^{a}, —O—C(O)NR^{a}R^{b}, —C(O)NR^{a}R^{b} group, R^{a} and R^{b} being as defined previously,
A represents
a hydrogen atom, or
a C₁-C₈ alkyl group, or
a C₂-C₈ alkenyl, or
a C₂-C₈ alkynyl, or
a C₃-C₆ cycloalkyl, or
an aryl, or
a heterocycle, or
a halogen atom or
a —(CH₂)ₙ—CN, —(CH₂)ₙ—CF₃, —(CH₂)ₙ—NO₂, —(CH₂)ₙ—OR^{a}, —(CH₂)ₙ—C(Me)₂OR^{a}, —(CH₂)ₙ—CHMeOR^{a}, —(CH₂)ₙ—SR^{a}, —(CH₂)ₙ—SO₂R^{a}, —CH₂—SeR^{a}, —(CH₂)ₙ—NR^{a}R^{b}, —C(O)R^{a}, —OC(O)NR^{a}R^{b}, —C(O)OR^{a}, —C(O)NR^{a}R^{b} group in which
(i) R^{a} and R^{b}, are as defined previously and
(ii) n represents an integer which can have any one of the values from 0 to 4, or also
a group corresponding to formula (V):
R^{c}-Q-(CH₂)ₘ (V)
in which
(i) m represents an equal integer which has any one of the values from 1 to 8; and
(ii) Q represents an oxygen atom or an —NR^{a} group in which R^{a} is as defined previously and
(iii) R^{c} represents
a. a hydrogen atom or
b. a C₁-C₆alkyl,
c. an aryl,
d. a heteroaryl,
e. a heterocycle,
f. an alkyl-C(O)—, the alkyl of which is C₁-C₆,
g. an aryl-C(O)—,
h. a heteroaryl-C(O)—,
i. a heterocycle-C(O)—,
j. a group represented by one of formulae (III) or (IV) 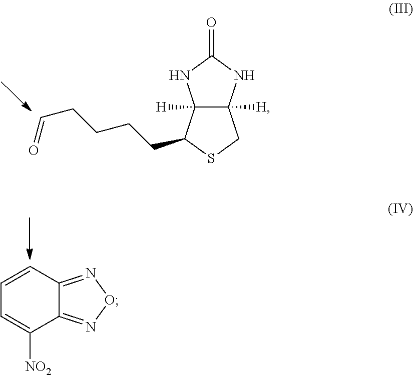
or
k. an —O—C(O)— group or
l. an —NR^{a}—C(O)— group in which R^{a} is as defined previously
If A with X together with the carbons to which they are attached represent a chain of formula (VI) 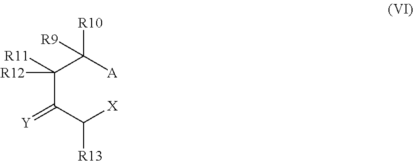
in which
Y represents a ketone (═O) group, an (═N—OH) oxime group or an alkyl (═N—O-alkyl) oxime group the alkyl group of which is C₁-C₆;
R₉ represents a hydrogen atom, a C₁-C₆ alkyl group, a halogen atom; or
R₉ and R₁₁ together form an additional carbon-carbon bond between the carbon atoms to which they are attached, or a C₃-C₆ cycloalkyl group;
R₁₀ represents a hydrogen atom, a halogen atom or an —OR^{a}, —SR^{a}, —CN, —NR^{a}R^{b} group, —R^{a} and —R^{b} being as defined previously;
R₁₁ represents a hydrogen atom, or a C₁-C₆ alkyl, C₃-C₆ cycloalkyl, aryl group or a halogen atom;
R₁₂ represents a hydrogen atom, or a C₁-C₆ alkyl group or a halogen atom or a —CN, —OR^{a}, —SR^{a}, —SeR^{a}, —C(O)—R^{a}, —C(O)OR^{a}, —NR^{a}R^{b}, —OC(O)NR^{a}R^{b} group, —R^{a} and —R^{b} being as defined previously; or
R₁₁ and R₁₂ together with the carbon to which they are attached, form a C₃-C₆ cycloakyl group;
R₁₃ represents a hydrogen atom or a C₁-C₆ alkyl group, or a halogen atom or a —CN, —CF₃, —NO₂, —OR^{a}, —SR^{a}, —SO₂R^{a}, —NR^{a}R^{b}, —C(O)—R^{a}, —OC(O)R^{a}, —OC(O)NR^{a}R^{b}, —C(O)OR^{a}, —C(O)NR^{a}R^{b} group or together with Z a carbon-carbon bond
then
Z represents a hydrogen atom, a hydroxyl group, a hydroxyamino group or together with R₁₃ a carbon-carbon bond and
R₁ represents a hydrogen atom or a —CH₃, —CH₂—CN, —CH₂—SR^{a}, —CH₂—SeR^{a} group or also a group corresponding to formula (VII) or (VIII) as follows:
—CH₂—W—R^{c} (VII) or
—C(O)—W—R^{c} (VIII)
in which
W represents an oxygen atom or an —NR^{a} group in which R^{a} is as defined previously or, a spacer arm constituted by an optionally substituted, linear or branched hydrocarbon chain comprising 2 to 20 carbon atoms and comprising moreover at least one heteroatom;
R^{c} being as defined previously;
as well as:
its SYN, ANTI geometrical isomers, when they exist,
its optical isomers (enantiomers, diastereoisomers), when they exist,
its addition salts with a pharmaceutically acceptable acid or base,
its hydrates and its solvates,
its prodrugs,
or one of its esters.

2. The liposome according to claim 1, which comprises at least one compound of formula I or a mixture of compounds of formula I, a phospholipid or a mixture of phospholipids and a pH stabilizing agent.

3. The liposome according to claim 2, further comprising a cryoprotective agent.

4. The liposome according to claim 1, wherein the compound of formula I is selected from the group consisting of:
cholest-4-en-3-one oxime,
cholest-4,24-dien-3-one oxime
cholestan-3-one oxime,
3-hydroxy-3,5-seco-4-norcholestan-5-one oxime,
1,4-cholestadien-3-one oxime,
2-methyl-cholest-4-en-3-one oxime,
2α-fluoro-cholest-4-en-3-one oxime,
4-methoxy-cholest-4-en-3-one oxime,
4-fluoro-cholest-4-en-3-one oxime,
6β-fluoro-cholest-4-en-3-one oxime,
19-hydroxy-cholest-4-en-3-one oxime,
19-biotinyloxy-cholest-4-en-3-one oxime,
25-((N-(+)-biotinoyl-N-methyl)amino)-27-norcholest-4-en-3-one oxime,
25-[methyl(7-nitro-2.1,3-benzoxadiazol-4-yl)amino]-27-norcholest-4-en-3-one oxime,
2,2-difluoro-cholest-4-en-3-one oxime,
2,6-difluoro-cholest-4-en-3-one oxime,
cholest-4-en-3,6-dione 3-oxime,
cholest-4,21-dien-3,6-dione 3-oxime,
24-ethyl-cholest-4-en-3,6-dione 3-oxime,
24-ethyl-cholest-4,21-dien-3,6-dione 3-oxime,
24-methyl-cholest-4,21-dien-3,6-dione 3-oxime,
3-[methyl(7-nitro-2.1,3-benzoxadiazol-4-yl)amino]-3,5-seco-4-norcholestan-5-one oxime,
2-hydroxy-2,5-seco-3,4-dinor-cholestan-5-one oxime,
3-[(N-(+)-biotinoyl-N-methyl)amino]-3,5-seco-4-norcholestan-5-one oxime,
25-fluoro-3-hydroxy-3,5-seco-4-norcholestan-5-one oxime,
3,5-seco-4-norcholestan-5-one oxime,
3-hydroxy-3,5-seco-4-norcholest-24-en-5-one oxime,
3-hydroxy-3,5-seco-3-trifluoromethyl-4-norcholestan-5-one oxime,
24β-ethyl-3-hydroxy-3,5-seco-4-norcholest-22-en-5-one oxime,
3-hydroxy-3-methyl-3,5-seco-4-norcholestan-5-one oxime,
3,3-dimethyl-3-hydroxy-3,5-seco-4-norcholestan-5-one oxime,
3-hydroxy-3,5-seco-4-norcholestan-5-one oxime,
cholest-4-en-3,6-dione dioxime,
cholest-4,24-dien-3,6-dione,
24-methyl-cholest-4,21-dien-3,6-dione dioxime,
24-ethyl-cholest-4-en-3,6-dione dioxime,
24-ethyl-cholest-4,21-dien-3,6-dione dioxime,
cholest-4-en-3-one,
cholest-4,24-dien-3-one,
3-hydroxy-3,5-seco-4-norcholestan-5-one,
3-hydroxy-3-methyl-3,5-seco-4-norcholestan-5-one,
3,3-dimethyl-3-hydroxy-3,5-seco-4-norcholestan-5-one, and
3-hydroxy-3,5-seco-4-norcholestan-5-one methyloxime.

5. The liposome according to claim 1, wherein the compound of formula I is present in a final liposomal solution, in a quantity ranging from 0.1 to 200 mg/mL.

6. The liposome according to claim 2, wherein the phospholipid is selected from the group consisting of phosphoacylglycerols (better known under the name of glycerophospholipids), inositophosphatides, phosphosphingolipids and phosphonosphingolipids or also phosphosaccharolipids, advantageously from the phospholipids which can be chosen from phosphatidylcholine, lysophosphatidylcholine, phosphatidylethanolamine, linoleylpalmitoylphosphatidylcholine, palmitoyloleoylphosphatidylcholine, oleoylpalmitoylphophatidylcholine, DHAstearoylphosphatidyl choline, DHA-rich phosphatidylcholine of avian origin, phosphatidylinositol, DHA-phosphatidylethanolamine, phosphatidylserine, sphingomyelin, a mixture of phospholipids of avian origin close to the composition of human milk, a mixture of phospholipids of soya origin close to the composition of human milk, lysophosphatidic palmitic or oleic acids, egg lysophosphatidylcholine containing palmitic and stearic acids at more than 90%, soya lysophosphatidylcholine, lysophosphatidylinositol, lysophosphatidylethanolamine, lysophosphatidylserine, a mixture of egg phospholipids containing phosphatidylcholine, phosphatidylethanolamine, phosphatidylserine, phosphatidylinositol and sphingomyelin, 1,2-dioleolyl-sn-glycero-3-phosphocholine (DOPC), 1,2-Dimyristoyl-sn-glycero-3-phosphorylcholine (DMPC), 1,2-dimyristoylphosphatidylglycerol (DMPG), egg L-α-phosphatidylcholine, soya L-α-phosphatidylcholine, and 1,2-distearoyl-sn-glycero-3-phosphoethanolamine-N-[methoxy(polyethylene glycol)-2000] (DSPE-PEG), yet more advantageously from egg or soya phospholipids, preferably egg phospholipids.

7. The liposome according to claim 2, wherein the phospholipid is present in a final liposomal solution in a quantity ranging from 10 to 300 mg/mL.

8. The liposome according to claim 2, wherein the pH stabilizing agent is a phosphate, benzoate, citrate, glutamate, lactate, ascorbate, tartrate, succinate, adipate, glycinate, malate, triethanolaminate, diethanolaminate, or tromethaminate buffer.

9. The liposome according to claim 3, wherein the cryoprotective agent is selected from the group consisting of glycerol, sucrose, dextrose, trehalose, glucose, maltose, mannose, lactose, mannitol, sorbitol, glycine, polyvinylpyrrolidone (PVP), polyvinylalcool (PVA), gelatin, alanine, lysine, polyethylene glycol, dextran, aerosil, fructose, and hydroxypropyl-β-cyclodextrin, preferentially from glycerol, sucrose or dextrose.

10. The liposome according to claim 3, wherein the cryoprotective agent is present in the liposomal formulation in a quantity ranging from 0.01 to 30% in solution.

11. A composition, comprising at least one liposome according to claim 1.

12-13. (canceled)

14. The composition according to claim 11, further comprising active ingredients other than the compounds of formula (I).

15. The liposome according to claim 5, wherein the compound of formula I is present in a final liposomal solution, in a quantity ranging from 0.1 mg/mL to below 1 mg/mL.

16. The liposome according to claim 5, wherein the compound of formula I is present in a final liposomal solution, in a quantity ranging from 0.1 mg/mL to below 5 mg/mL.

17. The liposome according to claim 5, wherein the compound of formula I is present in a final liposomal solution, in a quantity ranging from 0.1 mg/mL to below 10 mg/mL.

18. The liposome according to claim 7, wherein the phospholipid is present in a final liposomal solution in a quantity between 20 and 200 mg/mL.

19. The liposome according to claim 7, wherein the phospholipid is present in a final liposomal solution in a quantity between 50 and 150 mg/mL.

20. The liposome according to claim 10, wherein the cryoprotective agent is present in the liposomal formulation in a quantity between 0.1 and 20%.

21. The liposome according to claim 10, wherein the cryoprotective agent is present in the liposomal formulation in a quantity between 1 and 10%.

22. The liposome according to claim 2, wherein the pH stabilizing agent is a phosphate buffer.
